(19) United States
(12) Patent Application Publication
(10) Pub. No.: US 2010/0069411 A1
(43) Pub. Date: Mar. 18, 2010

(54) CANCER TREATMENT METHOD

(75) Inventors: Richard Buller, Hillsborough, CA (US); Mark Berger, Marion Station, PA (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SmithKline Beecham (Cork) Ltd.

(21) Appl. No.: 12/516,661

(22) PCT Filed: Nov. 9, 2007
(86) PCT No.: PCT/US07/84215

§ 371 Date: May 28, 2009

Related U.S. Application Data
(60) Provisional application No. 60/867,431, filed on Nov. 28, 2006.
(60) Provisional application No. 60/943,662, filed on Jun. 13, 2007.
(60) Provisional application No. 60/951,271, filed on Jul. 23, 2007.

(51) Int. Cl. A61K 31/519; A61P 35/00

(52) U.S. Cl. 514/265.1

(57) ABSTRACT

The present invention relates to a method of treating cancer in a mammal by administration of 4-quinazolinamines and at least one additional anti-neoplastic compound. In particular, the method relates to a methods of treating cancers by administration of N-{3-chloro-4-[(3-fluorobenzyl)oxy]phenyl}-6-[5-({[2-(methanesulphonyl)ethyl]amino}methyl)-2-furyl]-4-quinazolinamine and salts and solvates thereof in combination with at least one additional anti-neoplastic compound.

### BACKGROUND OF THE INVENTION

The present invention relates to a method of treating cancer in a mammal by administration of 4-quinazolinamines in combination with other anti-neoplastic compounds. In particular, the method relates to methods of treating cancers by administration of a combination of N-{3-chloro-4-[(3-fluorobenzyl)oxy]phenyl}-6-[5-({[2-(methanesulphonyl)ethyl]amino}methyl)-2-furyl]-4-quinazolinamine or salts or solvates thereof along with additional anti-neoplastic compounds.

Effective chemotherapy for cancer treatment is a continuing goal in the oncology field. Generally, cancer results from the deregulation of the normal processes that control cell division, differentiation and apoptotic cell death. Apoptosis (programmed cell death) plays essential roles in embryonic development and pathogenesis of various diseases, such as degenerative neuronal diseases, cardiovascular diseases and cancer. One of the most commonly studied pathways, which involves kinase regulation of apoptosis, is cellular signaling from growth factor receptors at the cell surface to the nucleus (Crews and Erikson, Cell, 74:215-17, 1993). In particular, cellular signalling from the growth factor receptors of the erbB family.

There is significant interaction among the erbB family that regulates the cellular effects mediated by these receptors. Six different ligands that bind to EGFR include EGF, transforming growth factor, amphiregulin, heparin binding EGF, betacellulin and epiregulin (Alroy & Yarden, FEBS Letters, 410:83-86, 1997; Burden & Yarden, Neuron, 18: 847-855, 1997; Klapper et al., Proc Natl Acad Sci, 4994-5000, 1999). Herugulins, another class of ligands, bind directly to HER3 and/or HER4 (Holmes et al., Science, 256:1205, 1992; Klapper et al., 1997, Oncogene, 14:2099-2109; Peles et al., Cell, 69:205, 1992). Binding of specific ligands induces homo- or heterodimerization of the receptors within members of the erbB family (Carraway & Cantley, Cell, 78:5-8, 1994; Lemmon & Schlessinger, TrendsBiochemSci, 19:459-463, 1994). In contrast with the other erbB receptor members, a soluble ligand has not yet been identified for HER2, which seems to be transactivated following heterodimerization. The heterodimerization of the erbB-2 receptor with the EGFR, HER3, and HER4 is preferred to homodimerization (Klapper et al., 1999; Klapper et al., 1997). Receptor dimerization results in binding of ATP to the receptor's catalytic site, activation of the receptor's tyrosine kinase, and autophosphorylation on C-terminal tyrosine residues. The phosphorylated tyrosine residues then serve as docking sites for proteins such as Grb2, Shc, and phospholipase C, that, in turn, activate downstream signaling pathways, including the Ras/MEK/Erk and the PI3K/Akt pathways, which regulate transcription factors and other proteins involved in biological responses such as proliferation, cell motility, angiogenesis, cell survival, and differentiation (Alroy & Yarden, 1997; Burgering & Coffer, Nature, 376:599-602, 1995; Chan et al., AnnRevBiochem, 68:965-1014, 1999; Lewis et al., AdvCanRes, 74:49-139, 1998; Liu et al., Genes and Dev, 13:786-791, 1999; Muthuswamy et al., Mol&CellBio, 19, 10:6845-6857, 1999; Riese & Stern, Bioessays, 20:41-48, 1998).

Several strategies including monoclonal antibodies (Mab), immunoconjugates, anti-EGF vaccine, and tyrosine kinase inhibitors have been developed to target the erbB family receptors and block their activation in cancer cells (reviewed in (Sridhar et al., Lancet, 4, 7:397-406, 2003)). Because ErbB2-containing heterodimers are the most stable and preferred initiating event for signaling, interrupting both erbB2 and EGFR simultaneously is an appealing therapeutic strategy. A series of 6-thiazolylquinazoline dual erbB-2/EGFR TK inhibitors that possess efficacy in pre-clinical models for cancer have been synthesized (Cockerill et al., BiorgMedChemLett, 11:1401-1405, 2001; Rusnak et al., CanRes, 61:7196-7203, 2001a; Rusnak et al., MolCanTher, 1:85-94, 2001b). GW572016 is a 6-furanylquinazoline, orally active, reversible dual kinase inhibitor of both EGFR and erbB2 kinases (Rusnak et al., 2001b). In human xenograft studies, GW572016 has shown dose-dependent kinase inhibition, and selectively inhibits tumor cells overexpressing EGFR or erbB2 (Rusnak et al., 2001b; Xia et al., Oncogene, 21:6255-6263, 2002).

Combination therapy is rapidly becoming the norm in cancer treatment, rather than the exception. Oncologists are continually looking for anti-neoplastic compounds which when utilized in combination provides a more effective and/or enhanced treatment to the individual suffering the effects of cancer. Typically, successful combination therapy provides improved and even synergistic effect over monotherapy.

### SUMMARY OF THE INVENTION

The present inventors have now identified novel cancer treatment methods which include administration of N-{3-chloro-4-[(3-fluorobenzyl)oxy]phenyl}-6-[5-({[2-(methanesulphonyl)ethyl]amino}methyl)-2-furyl]-4-quinazolinamine (GW572016) as well as salts and/or solvates thereof in combination with additional anti-neoplastic compounds.

In a first aspect of the present invention, there is provided a method of treating a susceptible cancer in a mammal, comprising: administering to said mammal therapeutically effective amounts of

(i) a compound of formula (I″)

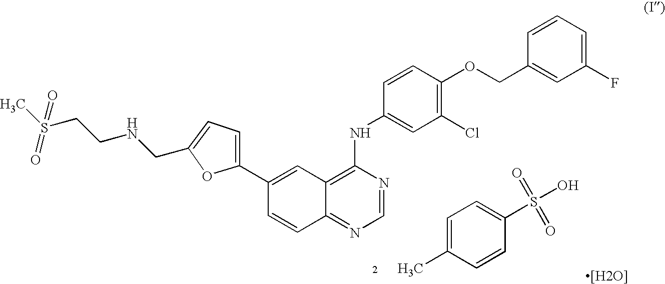

and

(ii) pemetrexed.

### DETAILED DESCRIPTION OF THE INVENTION

As used herein the term “neoplasm” refers to an abnormal growth of cells or tissue and is understood to include benign, i.e., non-cancerous growths, and malignant, i.e., cancerous growths. The term “neoplastic” means of or related to a neoplasm.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As is well known in the art, cancers or tumors are frequently metastatic, in that a first (primary) locus of cancerous tumor growth spreads to one or more anatomically separate sites. As used herein, reference to “a tumor” in a subject includes not only the primary tumor, but metastatic tumor growth as well. In a like manner reference to cancer or cancer treatment includes primary and metatatic cancer and treatment of the primary cancer and metastatic cancerous sites as well as prevention or recurrence of primary or metastatic cancer growth.

“EGFR”, also known as “erbB-1”, and “erbB-2” are protein tyrosine kinase transmembrane growth factor receptors of the erbB family. Protein tyrosine kinases catalyse the phosphorylation of specific tyrosyl residues in various proteins involved in the regulation of cell growth and differentiation (A. F. Wilks, Progress in Growth Factor Research, 1990, 2, 97-111; S. A. Courtneidge, Dev. Supp.l, 1993, 57-64; J. A. Cooper, Semin. Cell Biol., 1994, 5(6) 377-387; R. F. Paulson, Semin. Immunol., 1995, 7(4) 267-277; A. C. Chan, Curr. Opin. Immunol., 1996, 8(3), 394-401). The ErbB family of type I receptor tyrosine kinases includes ErbB1 (also known as the epidermal growth factor receptor (EGFR or HER1)), erbB2 (also known as Her2), erbB3, and erbB4. These receptor tyrosine kinases are widely expressed in epithelial, mesenchymal, and neuronal tissues where they play a role in regulating cell proliferation, survival, and differentiation (Sibilia and Wagner, Science, 269: 234 (1995); Threadgill et al., Science, 269: 230 (1995)). Increased expression of wild-type erbB2 or EGFR, or expression of constitutively activated receptor mutants, transforms cells in vitro (Di Fiore et al., 1987; DiMarco et al, Oncogene, 4: 831 (1989); Hudziak et al., Proc. Natl. Acad. Sci. USA., 84:7159 (1987); Qian et al., Oncogene, 10:211 (1995)). Increased expression of erbB2 or EGFR has been correlated with a poorer clinical outcome in some breast cancers and a variety of other malignancies (Slamon et al., Science, 235: 177 (1987); Slamon et al., Science, 244:707 (1989); Bacus et al, Am. J. Clin. Path, 102:S13 (1994)).

As used herein, the term “solvate” refers to a complex of variable stoichiometry formed by a solute (in this invention, compounds of formula (I) or a salt thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

As recited above the present invention is directed to cancer treatment methods which includes administration of N-{3-chloro-4-[(3-fluorobenzyl)oxy]phenyl}-6-[5-({[2-(methanesulphonyl)ethyl]amino}methyl)-2-furyl]-4-quinazolinamine (GW572016) as well as salts and/or solvates thereof in combination with other anti-neoplastic compounds.

The methods of cancer treatment disclosed herein includes administering a compound of formula (I):

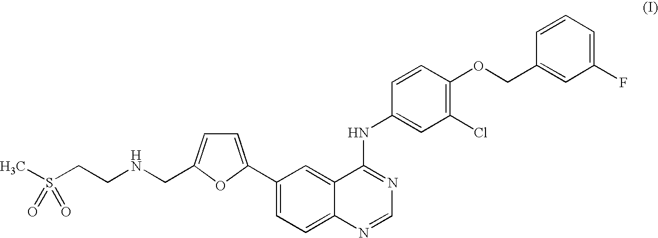

or salts or solvates thereof.

In another embodiment, the compound is a compound of formula (I′) which is the ditosylate salt of the compound of formula (I) or anhydrate or hydrate forms thereof. The ditosylate salt of the compound of formula (I) has the chemical name N-{3-chloro-4-[(3-fluorobenzyl)oxy]phenyl}-6-[5-({[2-(methanesulphonyl)ethyl]amino}methyl)-2-furyl]-4-quinazolinamine (GW572016) ditosylate and is also known as lapatinib.

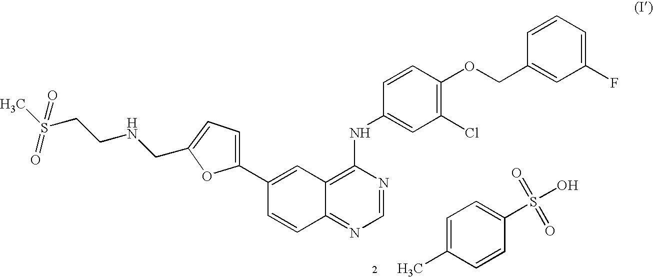

In one embodiment, the compound is the anhydrous ditosylate salt of the compound of formula (I′). In another embodiment, the compound is a compound of formula (I″) which is the monohydrate ditosylate salt of the compound of formula (I′).

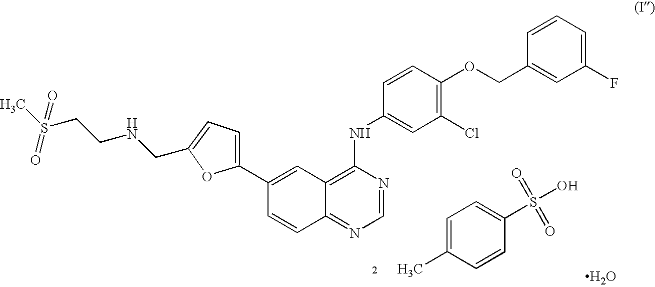

The free base, HCl salts, and ditosylate salts of the compound of Formula (I) may be prepared according to the procedures of International Patent Application No. PCT/EP99/00048, filed Jan. 8, 1999, and published as WO 99/35146 on Jul. 15, 1999, referred to above and International Patent Application No. PCT/US01/20706, filed Jun. 28, 2001 and published as WO 02/02552 on Jan. 10, 2002 and according to the appropriate Examples recited below. One such procedure for preparing the ditosylate salt of the compound of formula (I) is presented following in Scheme 1.

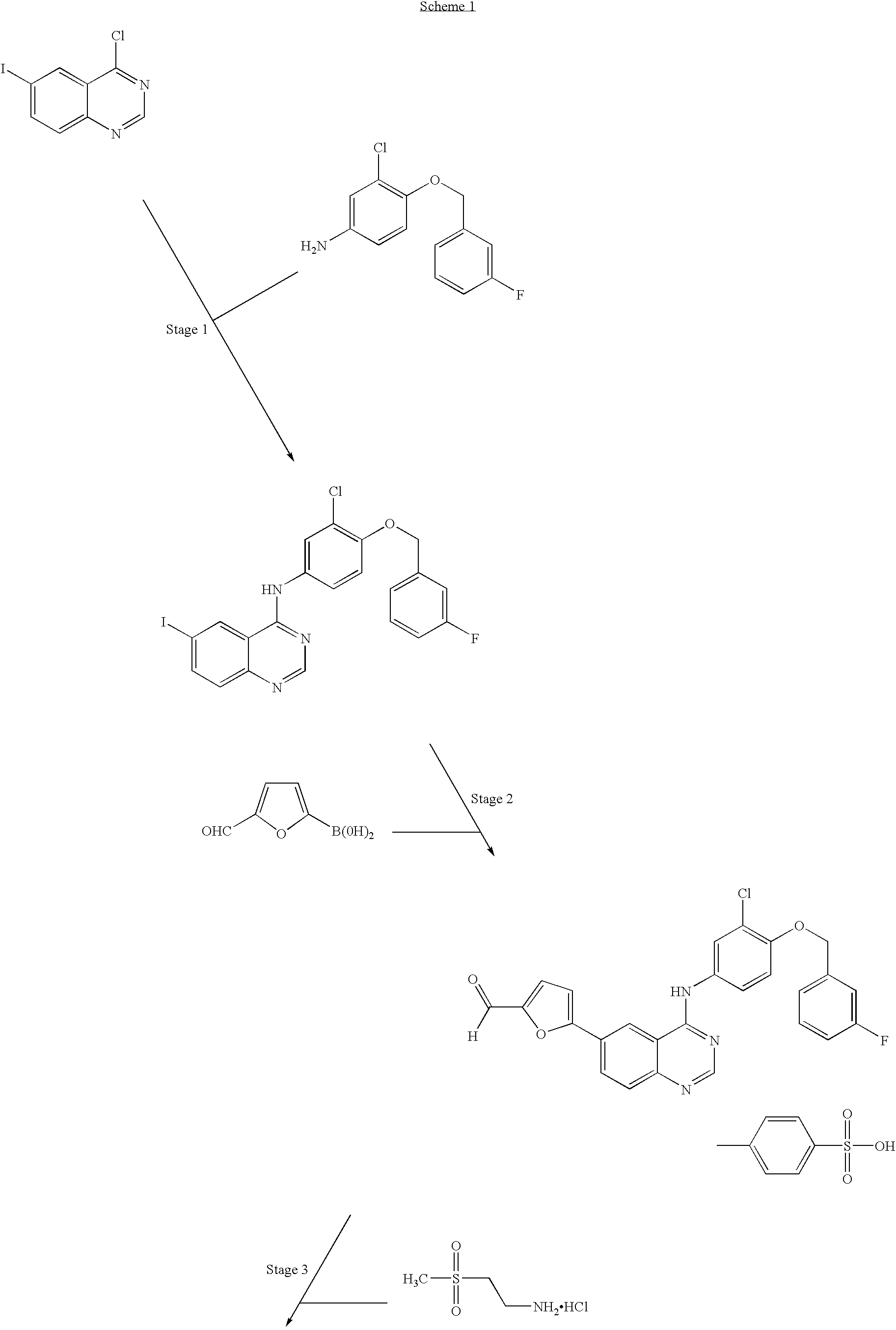

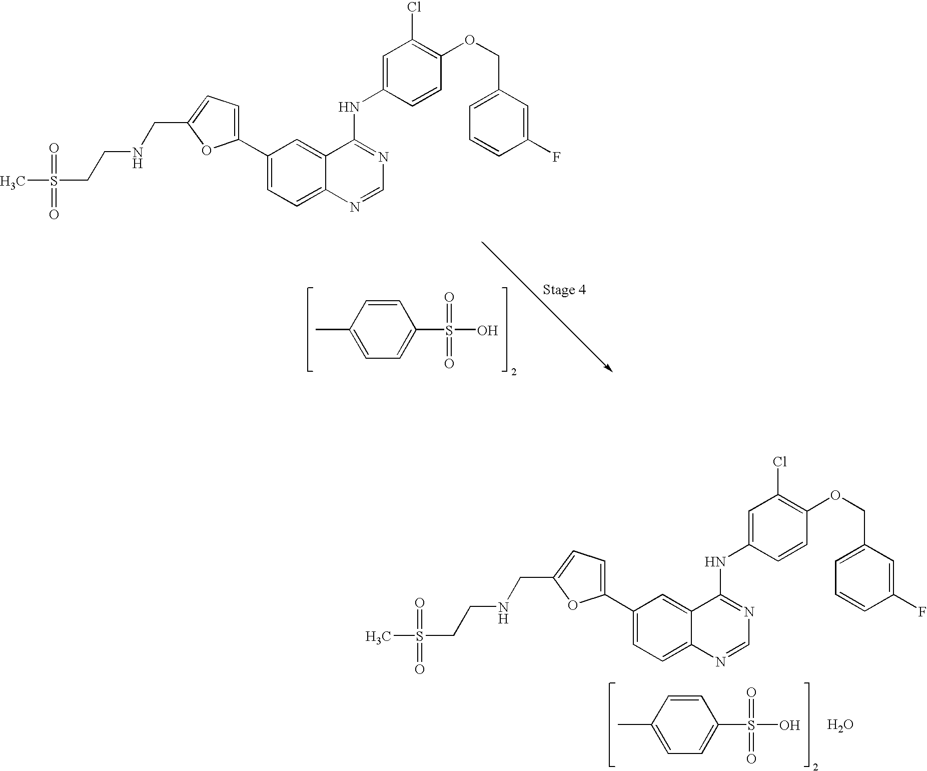

In scheme 1, the preparation of the ditosylate salt of the compound of formula (III) proceeds in four stages: Stage 1: Reaction of the indicated bicyclic compound and amine to give the indicated iodoquinazoline derivative; Stage 2: preparation of the corresponding aldehyde salt; Stage 3: preparation of the quinazoline ditosylate salt; and Stage 4: monohydrate ditosylate salt preparation.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in a compound of the present invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

In one embodiment, the cancer treatment method is a method of treating a susceptible cancer wherein the compound of formula (I″) is administered with pemetrexed. In one embodiment, the susceptible cancer is lung cancer. In one embodiment, the lung cancer overexpresses EGFR and/or erbB-2. In another embodiment, the susceptible cancer is non-small cell lung cancer. In one embodiment, the non-small cell lung cancer overexpresses EGFR and/or erbB-2.

Pemetrexed, L-glutamic acid, N-[4-[2-(2-amino-4,7-dihydro-4-oxo-1H-pyrrolo[2,3-d]pyrimidin-5-yl)ethyl]benzoyl-, disodium salt, heptahydrate; is commercially available as an injectable solution as ALIMTA®. Pemetrexed is indicated for the treatment of patients with locally advanced or metastatic non-small cell lung cancer after prior chemotherapy. Pemetrexed is an antifolate that exerts its antineoplastic activity by disrupting folate dependent metabolic processes essential for cell replication.

In one embodiment, the cancer treatment method is a method of treating a susceptible cancer wherein the compound of formula (I″) is administered with temozolamide. In one embodiment, the susceptible cancer is brain cancer. In one embodiment, the brain cancer overexpresses EGFR and/or erbB-2. In another embodiment, the susceptible cancer is glioblastoma. In one embodiment, the gliobastoma cancer overexpresses EGFR and/or erbB-2. In another embodiment, the susceptible cancer is astracytoma. In one embodiment, the astracytoma cancer overexpresses EGFR and/or erbB-2.

Temozolamide, 3,4-dihydro-3-methyl-4-oxoimidazo[5,1-d]-as-tetrazine-8-carboxamide; is commercially available as TEMODAR® capsules. Temozolamide is indicated for the treatment of adult patients with refractory anaplastic astrocytoma. Tomozolamide is converted at physiologic pH to the active compound 3-methyl-(triazenyl-1-yl)-imidazole-4-carboxamide (MTIC) which is thought to exert it cytotoxic effect through alkylation of DNA.

In one embodiment, the cancer treatment method is a method of treating a susceptible cancer wherein the compound of formula (I″) is administered with larotaxel. In one embodiment, the susceptible cancer is breast cancer. In one embodiment, the breast cancer overexpresses EGFR and/or erbB-2. In another embodiment, the susceptible cancer is pancreatic cancer. In one embodiment, the pancreatic cancer overexpresses EGFR and/or erbB-2.

Larotaxel, also known as XRP9881, is a semi-synthetic derivative of the taxane 10-deacetylbaccatin III being developed by Sanofi-Aventis. Larotaxel binds to tubulin, promoting microtubule assembly and stabilization and preventing microtubule depolymerization, thereby inhibiting cell proliferation.

In one embodiment, the cancer treatment method is a method of treating a susceptible cancer wherein the compound of formula (I″) is administered with pertuzumab. In one embodiment, the susceptible cancer is breast cancer. In one embodiment, the breast cancer overexpresses EGFR and/or erbB-2. In another embodiment, the susceptible cancer is ovarian cancer. In one embodiment, the ovarian cancer overexpresses EGFR and/or erbB-2.

Pertuzumab, also known as Omnitarg (2C4), is a humanized monoclonal antibody being developed by Genentech. Pertuzumab is a HER dimerization inhibitor which blocks the dimerization of HER2 dimerization pairs (HER2-HER1, HER2-HER3, HER2-HER4). It is thought that blockage of HER2 dimerization may prevent activation of intracellular signaling cascades, including MAPK and PI3K pathways, and thereby inhibit growth and proliferation of cancer cells.

In one embodiment, the cancer treatment method is a method of treating a susceptible cancer wherein the compound of formula I″ is administered with ixabepilone. In one embodiment, the susceptible cancer is breast cancer. In one embodiment, the breast cancer overexpresses EGFR and/or erbB-2. In another embodiment, the susceptible cancer is pancreatic cancer. In one embodiment, the pancreatic cancer overexpresses EGFR and/or erbB-2. In a further embodiment, the susceptible cancer is prostate cancer. In one embodiment, the prostate cancer overexpresses EGFR and/or erbB-2.

Ixabepilone (BMS 247550) is a semi-synthetic analog of epothilone B currently being developed by Bristol Myers Squibb. Ixabepilone is a microtubule stabilizing agent and promoter of microtubulin polymerization. It is thought such stabilization and polymerization of microtubulin leads to inhibition of the growth and proliferation of cancer cells.

In one embodiment, the cancer treatment method is a method of treating a susceptible cancer wherein the compound of formula I″ is administered with a heat shock protein 90 (HSP90) inhibitor. HSP90 is a chaperone protein which regulates the folding and stability, i.e., the conformational maturation and thereby the function of several signaling proteins that are associated with cancer. It is believed that interaction of HSP90 with such signaling proteins can lead to cancer cell proliferation. In one embodiment, HSP90 inhibitor is 17-Allylamino-17demethoxygeldanamycin (17AAG). In one embodiment, the susceptible cancer is breast cancer. In one embodiment, the breast cancer overexpresses EGFR and/or erbB-2.

In one embodiment, the cancer treatment method is a method of treating a susceptible cancer wherein the compound of formula (I″) is administered with oxaliplatin. In one embodiment, the susceptible cancer is colo-rectal cancer. In another embodiment, the susceptible cancer is gastric cancer or esophageal cancer. In another embodiment, the susceptible cancer is gastric cancer. In one embodiment, the gastric cancer overexpresses EGFR and/or erbB-2. In another embodiment, the susceptible cancer is esophageal cancer. In one embodiment, the esophageal cancer overexpresses EGFR and/or erbB-2.

Oxaliplatin, cis-[(1R,2R)-1,2-cyclohexanediamine-N,N′][oxalate(2-)-O,O′]platinum, is commercial available in injectable form as Eloxatin® from Sanofi-Aventis. Oxaliplatin is an organoplatinum complex in which a platinum atom is complexed with 1,2-diaminocyclohexane (DACH) and with an oxalate ligand as a leaving group. Under physiologic conditions oxaliplatin loses the oxalate ligand to form active derivatives which can covalently bind with macromolecules. Typically, interstrand and intrastrand platinum DNA crosslinks are formed which inhibit DNA replication and transcription. Oxaliplatin is approved for use in combination with 5-fluorouracil and leucovorin for the adjuvant treatment of stage III colon cancer patients whoses tumors have undergone complete resection or the treatment of advanced carcinoma in the colon or rectum.

In one embodiment, the cancer treatment method is a method of treating a susceptible cancer wherein the compound of formula (I″) is administered with oxaliplatin and 5-fluorouracil. In another embodiment, the susceptible cancer is gastric cancer or esophageal cancer. In another embodiment, the susceptible cancer is gastric cancer. In one embodiment, the gastric cancer overexpresses EGFR and/or erbB-2. In another embodiment, the susceptible cancer is esophageal cancer. In one embodiment, the esophageal cancer overexpresses EGFR and/or erbB-2.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil.

In one embodiment, the cancer treatment method is a method of treating a susceptible cancer wherein the compound of formula (I″) is administered with oxaliplatin and capecitabine. In another embodiment, the susceptible cancer is gastric cancer or esophageal cancer. In another embodiment, the susceptible cancer is gastric cancer. In one embodiment, the gastric cancer overexpresses EGFR and/or erbB-2. In another embodiment, the susceptible cancer is esophageal cancer. In one embodiment, the esophageal cancer overexpresses EGFR and/or erbB-2.

Capecitabine, 5′-deoxy-5-fluoro-N-[(pentyloxy)carbonyl]-cytidine; is commercially available as 150 or 500 mg tablets as XELODA®. Capecitabine is an orally administered pro-drug of 5′-deoxy-5-fluoruridine (5′-DFUR) which is converted into 5-fluorouracil in vivo. Capecitabine is indicated for treatment of metastatic breast cancer resistant to both paclitaxel and an anthracycline containing treatment regimen.

In one embodiment, the cancer treatment method is a method of treating a susceptible cancer wherein the compound of formula I″ is administered with a hedgehog pathway inhibitor. Activation of the Hedgehog pathway has been associated with several cancers. Patched (PTCH) is the receptor for Hedgehog ligands including Sonic hedgehog (SHh). PTCH receptor in the absence of Hedgehog ligands inhibits Smoothened (SMO) which is a G-coupled-like receptor. When a Hedgehog ligand binds to PTCH SMO is no longer inhibited the Hedgehog pathway is activated with increased activation of pathway transcription factors and initiation of a signaling cascade leading to increased cancer cell proliferation and metastasis. Inhibition of the Hedgehog pathway, for instance by SMO inhibition may lead to increased apoptosis and decreased invasiveness of cancer cells. In one embodiment, the Hedgehog Pathway inhibitor is the SMO inhibitor cyclopamine. In one embodiment, the susceptible cancer is prostate cancer. In one embodiment, the prostate cancer is androgen independent prostate cancer. In another embodiment, the cancer is breast, lung, brain or skin cancer. In another embodiment the cancer is basal cell carcinoma.

Combination therapies according to the present invention thus include the administration of the compound of formula (I″) as well as use of at least one other anti-neoplastic agent. Such combination of agents may be administered together or separately and, when administered separately this may occur simultaneously or sequentially in any order, both close and remote in time. The amounts of the compound of formula (I″) and the other pharmaceutically active agent(s) and the relative timings of administration will be selected in order to achieve the desired combined therapeutic effect.

Also contemplated in the present invention are pharmaceutical combinations including compounds of the Formula (I″) and at least one anti-neoplastic agent. Such compounds of formulae (I″) and the at least one anti-neoplastic agent are as described above and may be utilized in any of the combinations described above in the method of treating cancer of the present invention.

While it is possible that, for use in the cancer treatment methods of the present invention therapeutically effective amounts of a compound of formula (I″) as well as salts or solvates thereof, may be administered as the raw chemical, it is possible to present the active ingredient as a pharmaceutical composition. Accordingly, the invention further provides pharmaceutical compositions, which may be administered in the cancer treatment methods of the present invention. The pharmaceutical compositions include therapeutically effective amounts of a compound of formula (I″) or salts or solvates thereof, and one or more pharmaceutically acceptable carriers, diluents, or excipients. The carrier(s), diluent(s) or excipient(s) must be acceptable in the sense of being compatible with the other ingredients of the formulation and not deleterious to the recipient thereof.

Pharmaceutical formulations may be presented in unit dose forms containing a predetermined amount of active ingredient per unit dose. Such a unit depends, for example, on the condition being treated, the route of administration and the age, weight and condition of the patient. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

The compound of formula (I″) may be administered by any appropriate route. Suitable routes include oral, rectal, nasal, topical (including buccal and sublingual), vaginal, and parenteral (including subcutaneous, intramuscular, intravenous, intradermal, intrathecal, and epidural). It will be appreciated that the preferred route may vary with, for example, the condition of the recipient of the combination.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The agents for use according to the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Agents for use according to the present invention may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists that may be generated by means of various types of metered dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavoring agents.

Oral formulations for the ditosylate salt of the compound of Formula (I) are described in International Patent Application No. PCT/US2006/014447, filed Apr. 18, 2006, and published as WO 2006/113649 on Oct. 26, 2006.

As indicated, therapeutically effective amounts of a specific compound of formula (I″) is administered to a mammal. Typically, the therapeutically effective amount of one of the administered agents of the present invention will depend upon a number of factors including, for example, the age and weight of the mammal, the precise condition requiring treatment, the severity of the condition, the nature of the formulation, and the route of administration. Ultimately, the therapeutically effective amount will be at the discretion of the attendant physician or veterinarian.

The following examples are intended for illustration only and are not intended to limit the scope of the invention in any way.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

g (grams); mg (milligrams);

L (liters); mL (milliliters);

μL (microliters); psi (pounds per square inch);

M (molar); mM (millimolar);

N (Normal) Kg (kilogram)

i. v. (intravenous); Hz (Hertz);

MHz (megahertz); mol (moles);

mmol (millimoles); RT (room temperature);

min (minutes); h (hours);

mp (melting point); TLC (thin layer chromatography);

Tᵣ (retention time); RP (reverse phase);

DCM (dichloromethane); DCE (dichloroethane);

DMF (N,N-dimethylformamide); HOAc (acetic acid);

TMSE (2-(trimethylsilyl)ethyl); TMS (trimethylsilyl);

TIPS (triisopropylsilyl); TBS (t-butyldimethylsilyl);

HPLC (high pressure liquid chromatography);

THF (tetrahydrofuran); DMSO (dimethylsulfoxide);

EtOAc (ethyl acetate); DME (1,2-dimethoxyethane);

EDTA ethylenediaminetetraacetic acid

FBS fetal bovine serum

IMDM Iscove's Modified Dulbecco's medium

IMS Industrial Methylated Spirits

PBS phosphate buffered saline

RPMI Roswell Park Memorial Institute

RIPA buffer

RT room temperature

*150 mM NaCl, 50 mM Tris-HCl, pH 7.5, 0.25% (w/v)-deoxycholate, 1% NP-40, 5 mM sodium orthovanadate, 2 mM sodium fluoride, and a protease inhibitor cocktail.

Unless otherwise indicated, all temperatures are expressed in ° C. (degrees Centigrade). All reactions conducted under an inert atmosphere at room temperature unless otherwise noted.

GW572016F is lapatanib whose chemical name is N-{3-Chloro-4-[(3-fluorobenzyl)oxy]phenyl}-6-[5-({[2-(methane sulphonyl)ethyl]amino}methyl)-2-furyl]-4-quinazolinamine ditosylate monhydrate.

### Example 1

### Preparation of GW572016F

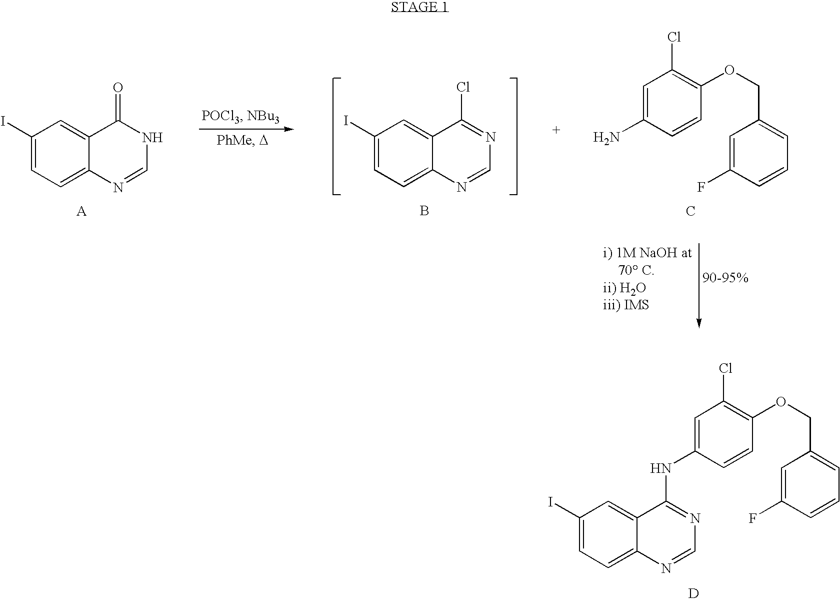

A stirred suspension of 3H-6-iodoquinazolin-4-one (compound A) in toluene (5 vols) was treated with tri-n-butylamine (1.2 eq.) at 20 to 25° C., then heated to 90° C. Phosphorous oxychloride (1.1 eq) was added, the reaction mixture was then heated to reflux. The reaction mixture was cooled to 50° C. and toluene (5 vols) added. Compound C (1.03 eq.) was added as a solid, the slurry was warmed back to 90° C. and stirred for 1 hour. The slurry was transferred to a second vessel; the first vessel was rinsed with toluene (2-vol) and combined with the reaction mixture. The reaction mixture was cooled to 70° C. and 1.0 M aqueous sodium hydroxide solution (16 vols) added dropwise over 1 hour to the stirred slurry maintaining the contents temperature between 68-72° C. The mixture was stirred at 65-70° C. for 1 hour and then cooled to 20° C. over 1 hour. The suspension was stirred at 20° C. for 2 hours, the product collected by filtration, and washed successively with water (3×5 vols) and ethanol (IMS, 2×5 vols), then dried in vacuo at 50-60° C.

Volumes are quoted with respect of the quantity of Compound A used. Percent yield range observed: 90 to 95% as white or yellow crystals.

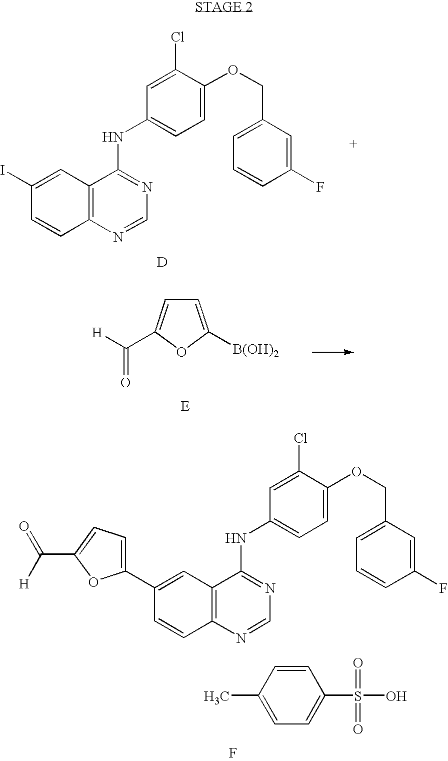

A mixture of N-{3-chloro-4-[(3-fluorobenzyl)oxy]phenyl}-6-iodo-4-quinazolinamine—compound D (1 wt), boronic acid—compound E (0.37 wt, 1.35 equiv), and 10% palladium on charcoal (0.028 wt, 50% water wet) was slurried in IMS (15 vol). The resultant suspension was stirred for 5 minutes, treated with di-isopropylethylamine (0.39 vol, 1.15 equiv) and then heated to ca 70° C. for ca 3 hours when the reaction was complete (determined by HPLC analysis). The mixture was diluted with tetrahydrofuran (THF, 15 vol) and then hot-filtered to remove the catalyst. The vessel was rinsed with IMS (2 vol).

A solution of p-toluenesulfonic acid monohydrate (1.5 wt, 4 equiv) in water (1.5 vol) was added over 5-10 minutes to the filtered solution maintained at 65° C. After crystallisation the suspension was stirred at 60°-65° C. for 1 hour, cooled to ca 25° C. over 1 hour and stirred at this temperature for a further 2 hours. The solid was collected by filtration, washed with IMS (3 vol) then dried in vacuo at ca 50° C. to give the compound F as a yellow-orange crystalline solid (isolated as the ethanol solvate containing approximately 5% w/w EtOH).

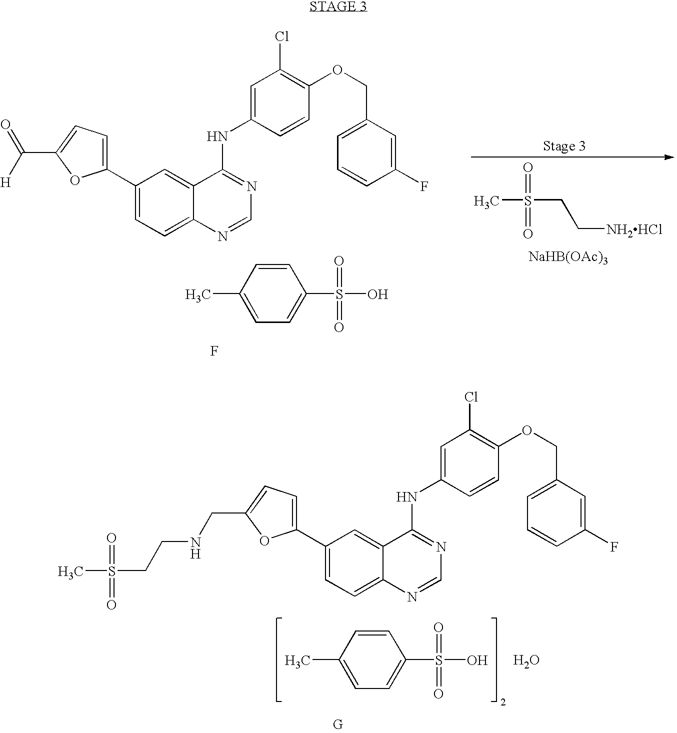

Compound F (1 wt) and 2-(methylsulfonyl)ethylamine hydrochloride (0.4 wt, 1.62 equiv.) were suspended in THF (10 vols). Sequentially, acetic acid (0.354 vol., 4 equiv.) and di-isopropylethylamine (DIPEA, 1.08 vol., 4.01 equiv.) were added. The resulting solution was stirred at 30°-35° C. for ca 1 hour then cooled to ca 22° C. Sodium tri-acetoxyborohydride (0.66 wt, 2.01 equiv.) was then added as a continual charge over approximately 15 minutes (some effervescence is seen at this point). The resulting mixture was stirred at ca 22° C. for ca 2 hours then sampled for HPLC analysis. The reaction was quenched by addition of aqueous sodium hydroxide (25% w/w, 3 vols.) followed by water (2 vols.) and stirred for ca 30 minutes (some effervescence was seen at the start of the caustic addition).

The aqueous phase was then separated, extracted with THF (2 vols) and the combined THF extracts were then washed twice with 25% w/v aqueous ammonium chloride solution (2×5 vols)². A solution of p-toluenesulfonic acid monohydrate (p-TSA, 0.74 wt, 2.5 equiv.) in water (1 vol)¹ was prepared, warmed to ca 60° C., and GW572016F (Compound G) (0.002 wt) seeds were added.

The THF solution of the free base of GW572016 was added to the p-TSA solution over at least 30 minutes, while maintaining the batch temperature at 60±3° C. The resulting suspension was stirred at ca 60° C. for 1-2 hours, cooled to 20-25° C. over an hour and aged at this temperature for ca 1 hr. The solid was collected by filtration, washed with 95:5 THF:Water (3×2 vols) and dried in vacuo at ca 35° C. to give GW572016F—compound G as a bright yellow crystalline solid. Expected yield 80% theory, 117% w/w.

¹ Minimum reaction volume ca 1 vol.
² Maximum reaction volume ca 17 vol.
# Corrected for assay.

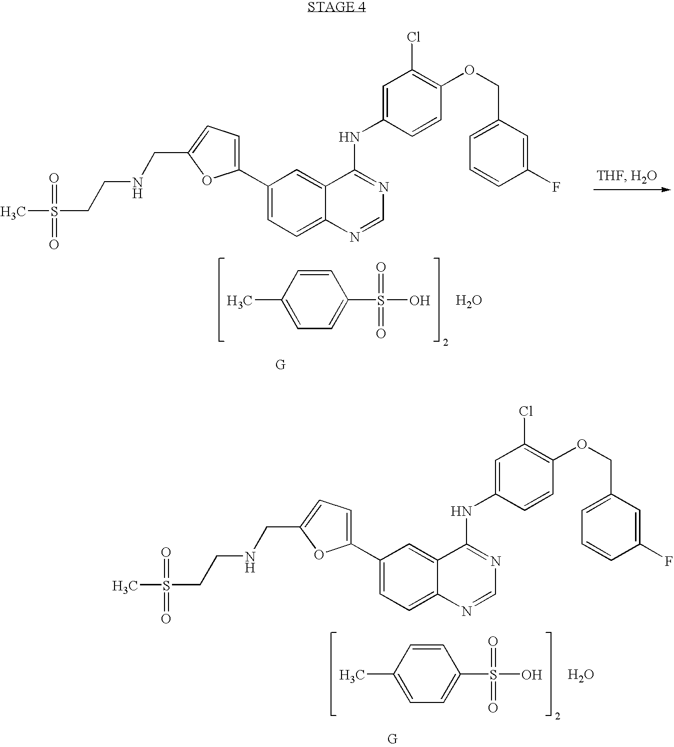

A suspension of the ditosylate monohydrate salt of N-{3-Chloro-4-[(3-fluorobenzyl)oxy]phenyl}-6-[5-({[2-(methane sulphonyl)ethyl]amino}methyl)-2-furyl]-4-quinazolinamino—compound G (1 wt), in tetrahydrofuran (THF, 14 vol) and water (6 vol) was heated to ca 55°-60° C. for 30 minutes to give a solution which was clarified by filtration and the lines washed into the crystallisation vessel with THF/Water (7:3 ratio, 2 vol). The resultant solution was heated to reflux and tetrahydrofuran (9 vol, 95% w/w azeotrope with water) was distilled off at atmospheric pressure.

The solution was seeded with N-{3-Chloro-4-[(3-fluorobenzyl)oxy]phenyl}-6-[5-({[2-(methane sulphonyl)ethyl]amino}methyl)-2-furyl]-4-quinazolinamine ditosylate monohydrate (0.002 wt). Once the crystallisation was established water (6 vol) was added while maintaining the reaction temperature above 55° C. The mixture was cooled to 5°-15° C. over ca 2 hours. The solid was collected by filtration, washed with tetrahydrofuran/water (3:7 ratio, 2 vol) then tetrahydrofuran/water (19:1 ratio, 2 vol) and dried in vacuo at 45° C. to give N-{3-Chloro-4-[(3-fluorobenzyl)oxy]phenyl}-6-[5-({[2-(methane sulphonyl)ethyl]amino}methyl)-2-furyl]-4-quinazolinamine ditosylate monohydrate—compound G as a bright yellow crystalline solid.

### Example 2

### Dosing with Lapatinib and Pemetrexed

The combination of pemetrexed and lapatinib was compared to each monotherapy using a method of statistical analysis designed to quantify the ability of a combination of two agents to inhibit tumor cell growth to an extent greater than the most potent single agent. Human lung carcinoma cells (Calu3, NCl-H322, NCl-H1650, and NCl-H1975: non-small cell lung cancer lines obtainable from the American Type Culture Collection) were cultured in a humidified incubator at 37° C. in 95% air, 5% CO₂ in RPMI 1640 containing 10% fetal bovine serum. Cells were assayed in a 96-well tissue culture plate (Falcon 3075) with the following plating densities: Calu-3, 10,000 cells/well, NCl-H322 5,000 cells/well, NCl-H1650, 10,000 cells/well, NCl H1975, 5,000 cells/well. Approximately 24 hours after plating cells were exposed to ten, two-fold serial dilutions of pemetrexed, lapatinib (10 micromolar to 0.020 micromolar for both agents) or the combination of the two agents. The final concentration of DMSO in all wells was 0.6%. Cells were incubated in the presence of compound for 3 days. Medium was then removed by aspiration. Cell biomass was estimated by staining cells with 90 microliters per well methylene blue (Sigma M9140, 0.5% in 1:1 ethanol:water), and incubation at room temperature for at least 30 minutes. Stain was removed, and the plates rinsed by immersion in deionized water and air-dried. To release stain from the cells 100 microliters of solubilization solution was added (1% N-lauroyl sarcosine, Sodium salt, Sigma L5125, in PBS), and plates were shaken gently for about 30 minutes. Optical density at 620 nM was measured on a microplate reader. Percent inhibition of cell growth was calculated relative to vehicle treated control wells. For each pair of monotherapy doses, the percent inhibition of the combination was compared to the more potent of the two monotherapies, and the probability that the combination was more potent than either monotherapy was defined (p-value). The p-values for all dose pairs were used to generate a T statistic. A T statistic less than −3 implies that the combination is better than the best single agent. A T statistic greater than 3 implies that the combination is worse than the best single agent. A T statistic greater than −3 and less than 3 implies the combination is the same as the best single agent.

### Results

| Cell Line | T Statistic | Comments |
|---|---|---|
| Calu3 | 1.86 | n = 1 |
| H322 | −7.90 | n = 3 |
| H1650 | −3.81 | n = 2 |
| H1975 | 0.26 | n = 3 |

What is claimed is:

1. A method of treating a susceptible cancer in a mammal, comprising: administering to said mammal therapeutically effective amounts of
(i) a compound of formula (I″) 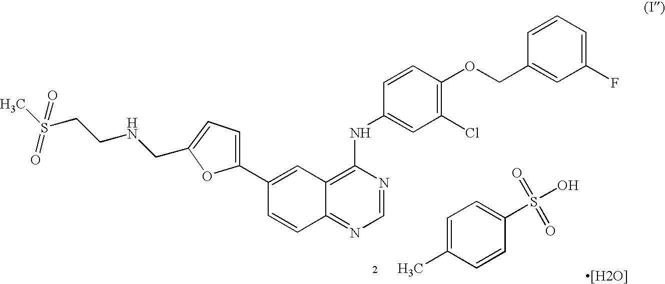
and
(ii) pemetrexed.
